(19) United States
(12) Patent Application Publication
(10) Pub. No.: US 2025/0246271 A1
(43) Pub. Date: Jul. 31, 2025

(54) INFORMATION PROCESSING DEVICE, INFORMATION PROCESSING METHOD, AND NON-TRANSITORY COMPUTER READABLE MEDIUM

(71) Applicant: ENEOS Corporation, Tokyo (JP)

(72) Inventor: Tasuku ONODERA, Tokyo (JP)

(73) Assignee: ENEOS Corporation, Tokyo (JP)

(21) Appl. No.: 19/071,046

(22) Filed: Mar. 5, 2025

Related U.S. Application Data
(63) Continuation of application No. PCT/JP2023/031492, filed on Aug. 30, 2023.

(30) Foreign Application Priority Data
Sep. 5, 2022 (JP) 2022-140872

(51) Int. Cl. G16C 20/50; G16C 20/70

(52) U.S. Cl. CPC G16C 20/50; G16C 20/70

(57) ABSTRACT

An information processing device includes a memory and a processor. The processor is configured to: define a molecular model representing a target molecular structure and a solid surface model; acquire an adsorption site and a position and angle at which the molecular model of a monomolecule approaches the solid surface model by executing a simulation of placing the molecular model of the monomolecule on the solid surface model; execute a simulation of making a plurality of the molecular models adsorb on a plurality of the adsorption sites; and acquire a structure and adsorption energy distribution when the molecular model of multiple molecules is adsorbed on the solid surface model.

### CROSS REFERENCE TO RELATED APPLICATIONS

This application is continuation application of International Application No. JP2023/031492, filed on Aug. 30, 2023, which claims priority to Japanese Application No. 2022-140872, filed on Sep. 5, 2022, the entire contents of which are incorporated herein by reference.

### FIELD

This disclosure relates to an information processing device, an information processing method, and a non-transitory computer readable medium.

### BACKGROUND

At an interface between a solid and gas or liquid, an adsorption phenomenon of molecules constituting the gas or liquid to a solid surface occurs. In general, there are two types of adsorption phenomena: physisorption due to van der Waals forces and chemisorption due to formation of chemical bonds such as covalent bonds. Among these, chemisorption is firm, and electronic and hybrid states of the adsorbed molecules change, which result in dissociating the chemical bonds the molecules possess or creating new chemical bonds with the solid surface.

Such chemisorption of molecules on the solid surface can significantly change properties and features possessed by the surface or interface, such as, for example, cohesiveness and dispersibility of solid particles in liquid, adhesiveness and removability of interfaces, and friction and abrasion, as well as substance conversion, such as catalytic reactions.

Therefore, when designing molecules that can express desired properties and features, it is common to use adsorption characteristics (structure, energy, and other characteristics) of a monomolecule as an indicator, and molecular simulation methods, such as, for example, a density functional theory (DFT), and techniques based on molecular dynamics are used to estimate the characteristics easily. In addition, a Monte Carlo method based on probability theory is commonly used to obtain multiple molecules adsorption characteristics.

However, when operations such as structure optimization are performed using methods based on quantum theory such as DFT, computing cost becomes very high, and it is difficult to screen many molecules in molecular designing. In addition, although the Monte Carlo method can consider the number of molecules adsorbed on a plurality of adsorption sites, it is difficult to simulate adsorption where a shape of the molecule changes significantly or chemical bonds are broken, because internal coordinates of the molecule are generally fixed.

### BRIEF DESCRIPTION OF THE DRAWINGS

FIG. 1 is a diagram schematically illustrating processes according to an embodiment.

FIG. 2 is a flowchart illustrating processes of an information processing device according to the embodiment.

FIG. 3 is a flowchart illustrating the processes of the information processing device according to the embodiment.

FIG. 4 is a diagram illustrating an example of adsorption according to the embodiment.

FIG. 5 is a diagram illustrating an example of a relationship between the number of adsorbed molecules and adsorption energy acquired by the information processing device according to the embodiment.

FIG. 6 is a diagram illustrating an example of an adsorption state acquired by the information processing device according to the embodiment.

FIG. 7 is a diagram illustrating an example of the relationship between the number of adsorbed molecules and adsorption energy acquired by the information processing device according to the embodiment.

FIG. 8 is a diagram illustrating an example of distribution of the number of molecules with respect to the adsorption energy acquired by the information processing device according to the embodiment.

FIG. 9 is a diagram illustrating an example of hardware implementation of the information processing device according to the embodiment.

### DETAILED DESCRIPTION

According to one embodiment, an information processing device includes a memory and a processor. The processor is configured to define a molecular model representing a target molecular structure and a solid surface model; acquire an adsorption site and a position and angle at which the molecular model of a monomolecule approaches the solid surface model by executing a simulation of placing the molecular model of the monomolecule on the solid surface model; execute a simulation of making a plurality of the molecular models adsorb on a plurality of the adsorption sites; and acquire a structure and adsorption energy distribution when the molecular model of multiple molecules is adsorbed on the solid surface model.

Embodiments of the present invention will be hereinafter described with reference to the drawings. The drawings and the description of the embodiments are presented by way of example only and are not intended to limit the present invention.

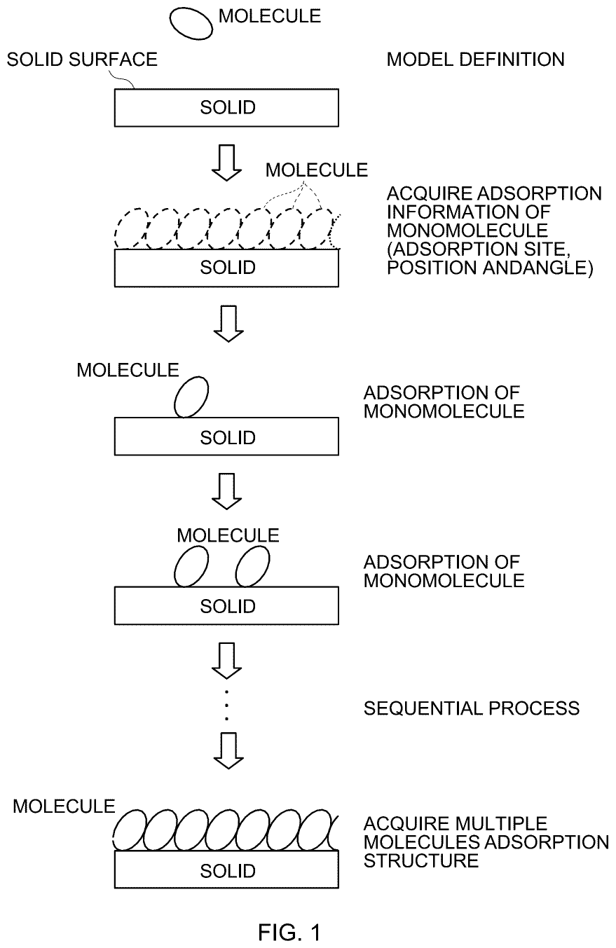

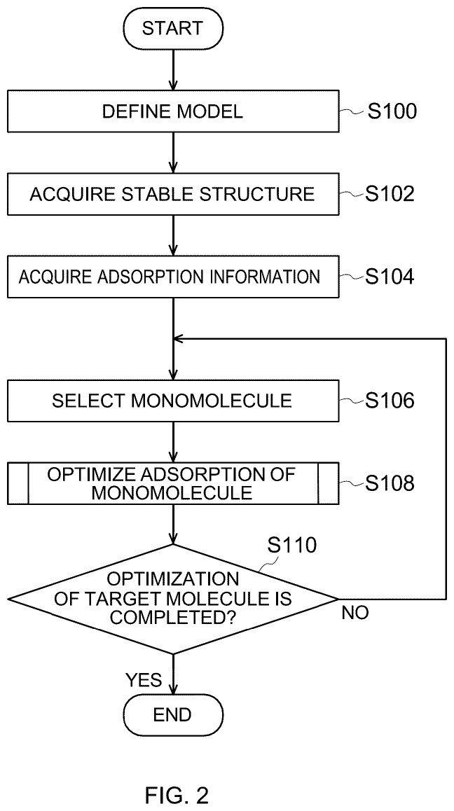

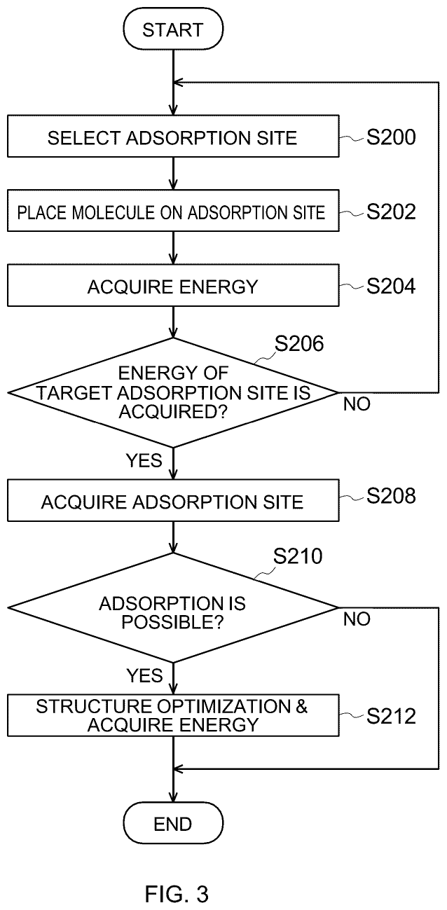

FIG. 1 is a diagram schematically illustrating processes according to an embodiment. In this embodiment, adsorption of each monomolecule is repeated sequentially to acquire a state of adsorption of multiple molecules on a solid surface. FIG. 2 and FIG. 3 are flowcharts illustrating the processes according to the embodiment. These diagrams are used to explain an example of the processes of an information processing device in this disclosure.

First, as illustrated in the flowchart in FIG. 2, the information processing device defines a model to be used for a simulation (S100). As illustrated in FIG. 1, for example, models of a monomolecule and a solid surface are defined.

Next, the information processing device uses the defined models to obtain a most stable structure where a monomolecule is adsorbed on a solid surface (S102). For example, the information processing device obtains the most stable structure using the solid surface model and the molecular model acquired in S100. This most stable structure can be obtained by using a structure optimization method such as a Broyden-Fletcher-Goldfarb-Shanno (BFGS) method, for example, to acquire information on a stable adsorption site when the molecular model of the monomolecule is adsorbed on the solid surface model, and an approach position and angle of the molecule.

The molecular model represents a target molecular structure. The solid surface model is a model for a target solid surface on which the molecular structure defined by the molecular model is adsorbed. The molecular model is a model that represents, for example, a molecular structure of propylene (C₃H₆), and the solid surface model is a model that represents, for example, an atomic arrangement of a (001) plane of iron (Fe).

The information processing device acquires information regarding adsorption of molecules on an object surface from the most stable structure acquired by the structure optimization, or other methods (S104). The information regarding adsorption is, for example, the information regarding the adsorption site when the monomolecule is adsorbed on the solid surface acquired by the structure optimization, or other methods, and the position and angle at which the molecule approaches this adsorption site. This adsorption may include both chemisorption and physisorption, for example.

This collected information regarding the adsorption of the monomolecules is schematically illustrated by dotted lines in a second row of FIG. 1, for example. The processes from S102 to S104 obtain the state where the monomolecule can be adsorbed, as schematically illustrated in this figure.

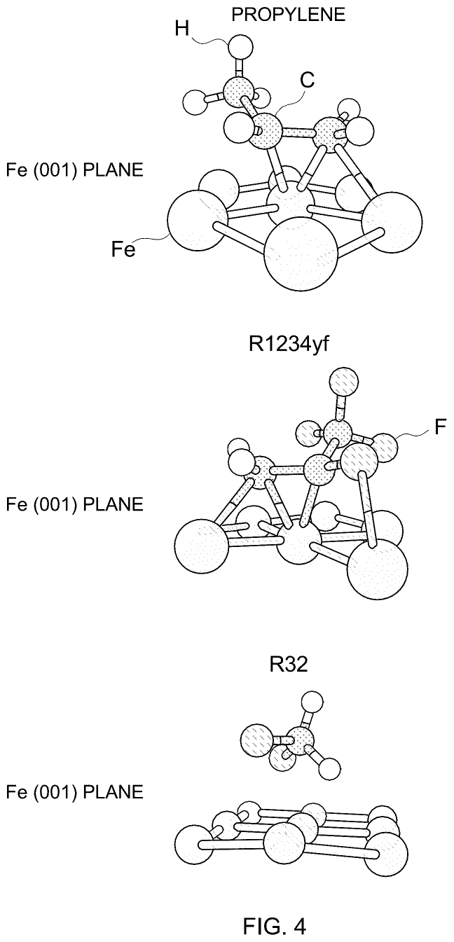

FIG. 4 lists several non-limiting examples of molecular adsorption on the (001) plane of Fe. In the top figure, propylene is chemisorbed on the Fe surface. In the middle figure, R1234yf (2,3,3,3-tetrafluoropropene) is chemisorbed on the Fe surface. In the bottom figure, R32 (difluoromethane) is physisorbed on the Fe surface.

The information processing device acquires such adsorption states. More concretely, information such as the adsorption site of the molecule on the surface model, and the position and angle of the molecule relative to the adsorption site (for example, an on-top positional relationship between a C═C double bond point in the molecule and the adsorbed surface molecule), is acquired by using the methods such as the structure optimization.

By the processes up to this point, the information processing device can acquire the state where the monomolecule is adsorbed on the solid surface. The information processing device uses this data to search for a structure where a plurality of molecules are adsorbed on the solid surface.

In acquiring the adsorption structure of multiple molecules, the information processing device performs the structure optimization in the case when the multiple molecules is adsorbed on the solid surface by making the monomolecule adsorb on the solid surface and sequentially searching for adsorption of the next monomolecule on the solid surface including the adsorbed molecules.

The information processing device searches for the most stable structure in the case when the multiple molecules is adsorbed using, for example, a neural network potential (NNP) method. When the structure optimization is acquired for a plurality of types of molecules, the information processing device searches for the most stable structure for each type of monomolecule. This most stable structure is information on the adsorption site for the monomolecule and the approach position and angle of the molecule acquired by the structure optimization, or other methods, but there are a plurality of similar adsorption sites on the solid surface. In other words, this most stable structure is extracted as a candidate for the adsorption site of one molecule illustrated by the molecular model.

NNP is a method of acquiring physical property values from an atomic arrangement using a trained model that outputs the physical property values when data such as the atomic arrangement are input. As a non-limiting example, the information processing device acquires the physical property values such as energy and force for an arbitrary position, angle, and the like of the molecule relative to the solid surface by inputting a solid surface model and the molecular model into the trained model used for NNP to obtain the structure where the monomolecule is adsorbed on the solid surface. Preferably, the information processing device obtains the most stable structure using methods such as structure optimization. That is, the information processing device applies the output (physical property values) of the trained model used for NNP as an interatomic potential.

The information processing device selects a model of the monomolecule (S106).

Next, the information processing device acquires data on how the selected monomolecule is adsorbed on the solid surface by performing optimization (S108). The flowchart in FIG. 3 illustrates this process of S108 in detail.

The information processing device selects the adsorption site for the selected monomolecule (S200). The selection of the adsorption site may be performed randomly. As a non-limiting example, an array of the plurality of adsorption sites acquired in S102 that are re-sorted by a random number may be acquired, and adsorption for the selected monomolecule may be attempted by a simulation according to the order of this array. As another example, the plurality of adsorption sites acquired in S102 may be randomly acquired, and sequential simulations may be performed.

The information processing device places the selected monomolecule on the selected adsorption site (S202). At the time of placement, the information processing device may randomly change the adsorption position and adsorption angle of the monomolecule with respect to the adsorption site. The information processing device may, for example, rotate the molecular model around an axis normal to the solid surface and randomly set it to a selectable angle.

The information processing device acquires the energy at the position and angle of the molecule relative to the acquired adsorption site (S204). The information processing device may use the NNP method to acquire this energy value. The information processing device may reject the adsorption of the molecule at the selected position and angle on the adsorption site at this time when a distance to the already adsorbed molecule is a predetermined distance or less. This determination may be based on a condition such that, for example, a distance to an adjacent molecule is 1 Å or less.

The information processing device determines whether the energy of the adsorption site to be the selection target is acquired (S206). When the acquisition of the energy of the selectable adsorption site has not been completed (S206: NO), the processes from S200 are repeated.

This process is not limited to sequential processes, but may also include parallel operation up to the acquisition of the energy values of the plurality of adsorption sites for the selected molecule. For example, by using a processor such as a GPU that can perform SIMD operation with a plurality of arithmetic cores, the information processing device can also acquire data regarding the plurality of adsorption sites for the molecule in parallel.

After acquiring the data of the selectable adsorption sites for the molecule, the information processing device acquires an optimal adsorption site (S208). For example, the information processing device acquires the adsorption site with a lowest energy value as the site where the molecule is adsorbed.

When there is more than one adsorption site with the lowest energy value, the information processing device may, for example, acquire the adsorption site with the lowest energy value, which is acquired for the first time among them, as the site where the molecule is adsorbed. In the case of parallel operation, the information processing device may, for example, assume the adsorption site with the low energy value acquired earliest in a randomly sorted order to be the site where the molecule is adsorbed, or the device may randomly acquire the site where the molecule is adsorbed from the adsorption sites with the lowest energy value.

The information processing device may determine whether the molecule can be adsorbed on the acquired adsorption site (S210). This process may be omitted when the state such as rejection, or other states has been determined in advance.

When the adsorption is possible (S210: YES), the information processing device performs the structure optimization of the focused molecule by acquiring the physical property values including the energy value and/or force by NNP (S212).

When the structure optimization in S212 is completed, the optimized structure is recorded, and when the adsorption is not possible (S210: NO), the optimization process for the currently focused molecule in FIG. 2 is completed without performing the structure optimization for the focused molecule (S108).

The information processing device determines whether the optimization of the target molecule has been completed (S110). When there are molecules among candidate molecules that have not yet been optimized for adsorption (S110: NO), the information processing device selects a molecule other than those that have been optimized for adsorption so far (next molecule) (S106) and performs the optimization for the adsorption of the selected monomolecule (S108).

When focusing on a second or subsequent molecule, the information processing device performs the structure optimization, such as searching for the adsorption site for the focused molecule, based on the adsorbed structures that have been acquired in previous iterations. In this case, the information processing device may not target the adsorption site where the molecule has already been adsorbed. When the parallel operation is executed, the data acquired for the adsorption sites where molecules have already been adsorbed in previous iterations may be dismissed after the processes are performed for all adsorption sites.

The information processing device sequentially repeats the adsorption between the molecular model of the monomolecule and the solid surface model and performs a search for the adsorption state between the molecular model of multiple molecules and the solid surface model.

When the information processing device determines that the molecule is adsorbed on the solid surface in the process of S108, this adsorption energy may be stored in a storage circuit. The information processing device can acquire distribution of the adsorption energy after the structure optimization is completed.

Thus, the information processing device of the embodiment defines the molecular model representing the target molecular structure and the solid surface model and executes the simulation where the molecular model of the monomolecule is placed on the solid surface model to acquire the adsorption site and the position and angle at which the molecular model of the monomolecule approaches the solid surface model.

As described in the flowchart above, the information processing device can achieve a simulation of the adsorption of the multiple molecules on the solid surface by executing the simulation of making the plurality of molecular models adsorb on the plurality of adsorption sites. The information processing device may acquire the stable adsorption sites, as well as the positions and angles by, for example, making the molecular model of the monomolecule adsorb on the plurality of adsorption sites. The information processing device may, for example, acquire the most stable structure on which the molecular model of the multiple molecules is adsorbed when chemical bond formation, dissociation, or change of the hybrid state occurs at the timing of adsorption of the molecular model on the solid surface model. In this simulation, it is possible to acquire the stable structure of the multiple molecules even when, for example, the formation or dissociation of chemical bonds of the molecules on the solid surface, or a change in their hybrid state occurs, since the physical property values such as energy using NNP are used.

The information processing device may, for example, randomly extract the adsorption site from the plurality of adsorption sites, and make the molecular model of the monomolecule adsorb on the extracted adsorption site in the simulation. By randomly selecting the molecules and adsorption sites and acquiring the physical property values by using NNP in the case where each molecule is placed on the solid surface, it is possible to acquire fast and accurate physical property values and perform robust structure optimization. As a result, structure optimization where the multiple molecules is adsorbed on the solid surface can be achieved with high speed and high accuracy.

The use of NNP allows users to achieve highly accurate simulations simply by setting up appropriate solid surface models and molecular models, that is, without setting up detailed parameters, and the like.

The information processing device can also perform the above structure optimization multiple times in parallel or sequentially. The information processing device may, for example, execute the simulation of making the molecular model adsorb by performing parallel operations regarding the plurality of adsorption sites. Alternatively, the information processing device may sequentially repeat the simulation of making the molecular model of the monomolecule adsorb on the plurality of adsorption sites to acquire the stable adsorption site, as well as the position and angle, for the plurality of monomolecular models.

According to the embodiment, the information processing device can acquire the structure and adsorption energy distribution when the molecular model of the multiple molecules is adsorbed on the solid surface model. By using a plurality of results, the information processing device can acquire the adsorption energy distribution statistically as well. As a result, it is also possible to predict how each molecule is adsorbed on the solid surface by focusing on the adsorption energy distribution.

From this, the information processing device can also predict quality, such as strength and adhesiveness of a film resulting from the aggregation and adsorption of molecules, and/or physical properties regarding the surface, such as surface energy and friction resistance brought by the quality, and/or performance by analyzing the adsorption energy distribution of the molecules, their frequency, and/or a degree of concentration of molecules.

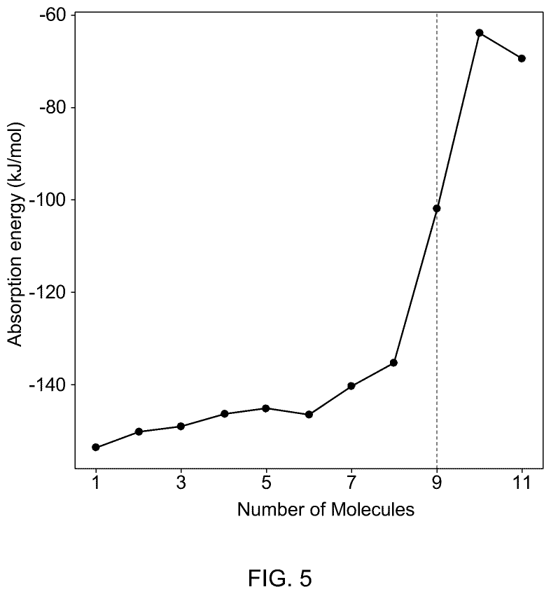

FIG. 5 is a diagram illustrating an ensemble average of multiple simulations of propylene adsorption on a 17 Å×17 Å Fe (001) plane performed by the above information processing device. The horizontal axis represents the number of adsorbed molecules, and the vertical axis represents the adsorption energy (the lower the value, the stronger the adsorption state).

As illustrated in this figure, when the number of adsorbed molecules reaches nine or more, the adsorption state rapidly becomes weak. This is because the number of placements where molecules are chemisorbed on the solid surface model decreases and shifts to second-layer adsorption or physisorption.

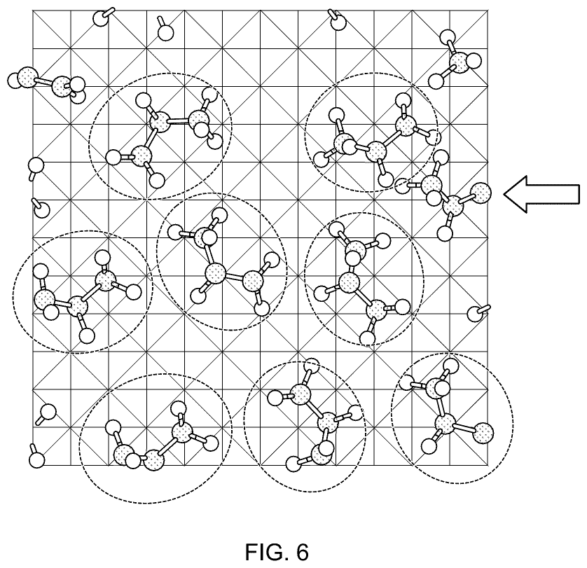

FIG. 6 is a diagram illustrating an example of the adsorption states of the molecules on the solid surface in the same state acquired by the information processing device. In this figure, each molecule surrounded by a dotted line is chemisorbed on the solid surface. In such an adsorption state, for example, a molecule that is adsorbed but cannot be chemisorbed properly or, even if the molecule is chemisorbed, the strength of adsorption is weak, as indicated by an arrow on a right edge, begins to occur. Thus, it can be seen that the graph in FIG. 5 appropriately illustrates the occurrence of the molecule whose adsorption strength weakens as the number of molecules increases.

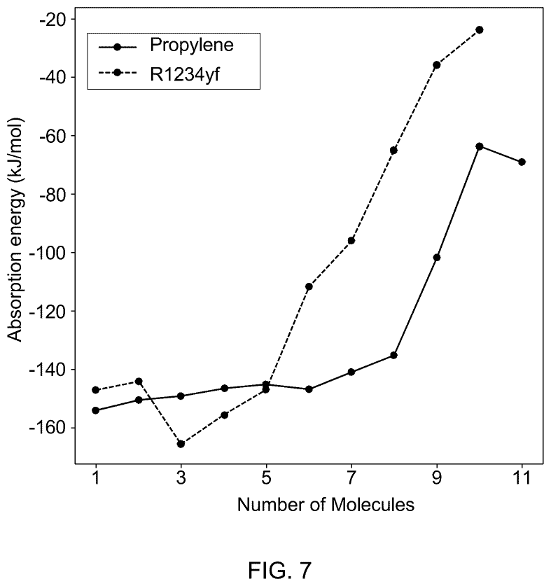

FIG. 7 is a diagram illustrating the adsorption state of R1234yf acquired by the information processing device in the same way, together with propylene in FIG. 5, superimposed on each other. When the number of adsorbed molecules is small such as in a range of one to four, for example, the adsorption strengths of propylene and R1234yf are the same or the adsorption strength of R1234yf is slightly stronger since both are chemisorbed. This is because R1234yf has a Fe—F bond as well as a Fe—C bond, which is the chemisorption starting from a C═C double bond similar to that of propylene.

On the other hand, in R1234yf, the adsorption strength gradually weakens from about six molecules, because the size of the molecule in R1234yf is larger than that in propylene, making chemisorption on a first layer more difficult at the smaller number of molecules.

As illustrated in these figures, it can be seen that the information processing device in this disclosure can acquire the appropriate adsorption states.

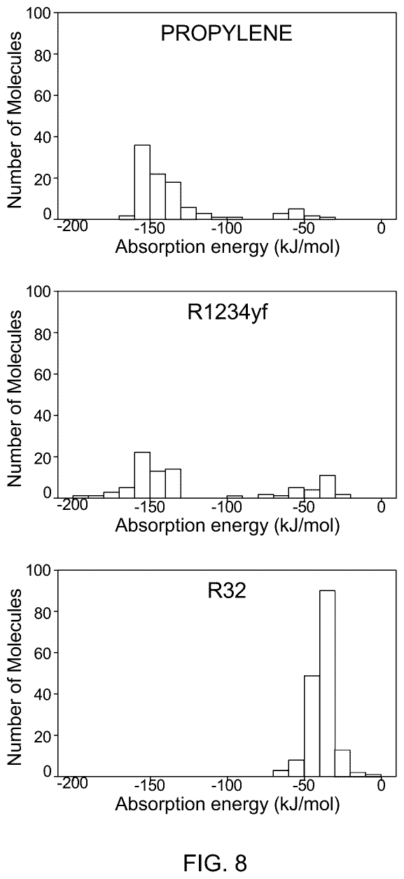

FIG. 8 is a diagram illustrating an example of distribution of the number of molecules with respect to the adsorption energy acquired by the information processing device in this disclosure. These figures show the adsorption energies on the Fe (001) plane when the molecules are propylene, R1234yf, and R32, starting from the top figure. The number of adsorbed molecules per ensemble is 10 molecules for propylene, 8.7 molecules for R1234yf, and 16.3 molecules for R32.

As shown in the figures, the distribution of strong adsorption, which is chemisorption, and the distribution of weak adsorption, which is physisorption, properly appear for propylene and R1234yf. On the other hand, the distribution showing only weak physisorption appears appropriately, for R32, which is not chemisorbed.

Thus, it can be seen that the information processing device in this disclosure can appropriately acquire strong adsorption and weak adsorption in the simulation.

The trained models of above embodiments may be, for example, a concept that includes a model that has been trained as described and then distilled by a general method.

Some or all of each device (the information processing device) in the above embodiment may be configured in hardware, or information processing of software (program) executed by, for example, a Central Processing Unit (CPU), a Graphics Processing Unit (GPU). In the case of the information processing of software, software that enables at least some of the functions of each device in the above embodiments may be stored in a non-volatile storage medium (non-volatile computer readable medium) such as Compact Disc Read Only Memory (CD-ROM) or Universal Serial Bus Memory (USB memory), and the information processing of software may be executed by loading the software into a computer. In addition, the software may also be downloaded through a communication network. Further, entire or a part of the software may be implemented in a circuit such as an Application Specific Integrated Circuit (ASIC) or Field Programmable Gate Array (FPGA), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

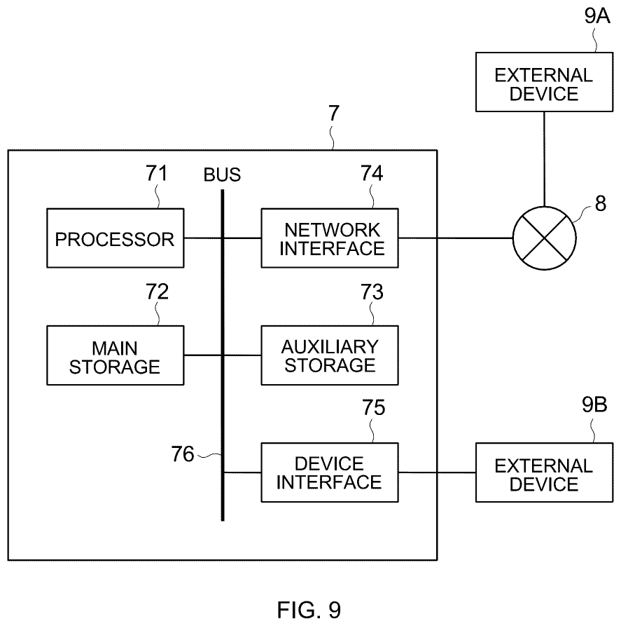

FIG. 9 is a block diagram illustrating an example of a hardware configuration of each device (the information processing device) in the above embodiments. As an example, each device may be implemented as a computer 7 provided with a processor 71, a main storage device 72 (a memory), an auxiliary storage device 73 (a memory), a network interface 74, and a device interface 75, which are connected via a bus 76.

The computer 7 of FIG. 9 is provided with each component one by one but may be provided with a plurality of the same components. The software may be installed on a plurality of computers, and each of the plurality of computer may execute the same or a different part of the software processing. In this case, it may be in an aspect of distributed computing where each of the computers communicates with each of the computers through, for example, the network interface 74 to execute the processing. That is, each device (the information processing device) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in an aspect of parallel computing by one or more computers.

The processor 71 may be an electronic circuit (such as, for example, a processor, processing circuity, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or a non-volatile memory. The storage device for storing various data or the like in each device (the information processing device) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73. For example, the processor may refer the data relating to the trained model in order to configure the trained model to implement at least a part of the process in this disclosure. The storage device may, for example, store data relating to the trained model which is inputted the information on the molecular and outputs at least one of the physical properties. For example, the processor executes a simulation of adsorbing a plurality of molecular models to a plurality of adsorption sites using the trained model. The trained model is, for example, a model used in Neural Network Potential (NNP). For example, the physical property value includes at least the energy or the force of the molecule.

In the case of each device (the information processing device) in the above embodiments is configured by at least one storage device (memory) and at least one processor connected/coupled to/with this at least one storage device, the at least processor may be connected to a single storage device. Or the at least storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device) in the above embodiments. That is, the computer 7 may transmit a part of or all of processing results to the external device 9A or the external device 9B, or receive a part of or all of processing results from the external device 9A or the external device 9B.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising:
a memory; and
a processor configured to:
define a molecular model representing a target molecular structure and a solid surface model;
acquire an adsorption site and a position and angle at which the molecular model of a monomolecule approaches the solid surface model by executing a simulation of placing the molecular model of the monomolecule on the solid surface model;
execute a simulation of making a plurality of the molecular models adsorb on a plurality of the adsorption sites; and
acquire a structure and adsorption energy distribution when the molecular model of multiple molecules is adsorbed on the solid surface model.

2. The information processing device according to claim 1, wherein the memory stores data regarding a trained model that outputs physical property values upon input of molecular information, and the processor acquires information regarding the trained model from the memory to constitute the trained model; and executes a simulation of making the plurality of molecular models adsorb on the plurality of adsorption sites using the trained model.

3. The information processing device according to claim 2, wherein the trained model is a model used for NNP (neural network potential), and the physical property values include at least energy or force of a molecule.

4. The information processing device according to claim 3, wherein the processor acquires the adsorption site that is stable, as well as the position and the angle by making the molecular model of a monomolecule adsorb on the plurality of adsorption sites.

5. The information processing device according to claim 4, wherein the processor executes a simulation of making the molecular model adsorb by performing parallel operations regarding the plurality of adsorption sites.

6. The information processing device according to claim 4, wherein the processor sequentially repeats the simulation for a molecule of acquiring the adsorption site that is stable, as well as the position and the angle by making the molecular model of the monomolecule adsorb on the plurality of adsorption sites for a plurality of monomolecular models.

7. The information processing device according to claim 4, wherein the processor randomly extracts the adsorption site from the plurality of adsorption sites, and makes the molecular model of the monomolecule adsorb on the extracted adsorption site.

8. The information processing device according to claim 1, wherein the processor acquires a most stable structure on which the molecular model of multiple molecules is adsorbed when chemical bond formation, dissociation, or change of a hybrid state occurs at a timing of adsorption of the molecular model on the solid surface model.

9. An information processing method comprising:
defining, by a processor, a molecular model representing a target molecular structure and a solid surface model;
acquiring, by the processor, an adsorption site and a position and angle at which the molecular model of a monomolecule approaches the solid surface model by executing a simulation of placing the molecular model of the monomolecule on the solid surface model;
executing, by the processor, a simulation of making a plurality of the molecular models adsorb on a plurality of the adsorption sites; and
acquiring, by the processor, a structure and adsorption energy distribution when the molecular model of multiple molecules is adsorbed on the solid surface model.

10. A non-transitory computer readable medium storing program causing a processor to execute an information processing method, the information processing method comprising:
defining a molecular model representing a target molecular structure and a solid surface model;
acquiring an adsorption site and a position and angle at which the molecular model of a monomolecule approaches the solid surface model by executing a simulation of placing the molecular model of the monomolecule on the solid surface model;
executing a simulation of making a plurality of the molecular models adsorb on a plurality of the adsorption sites; and
acquiring a structure and adsorption energy distribution when the molecular model of multiple molecules is adsorbed on the solid surface model.
